(19) United States
(12) Patent Application Publication
(10) Pub. No.: US 2008/0317856 A1
(43) Pub. Date: Dec. 25, 2008

(54) Compositions Containing A Capillary -Active System With Application -Relevant Differentiability, And The Use Thereof

(76) Inventors: Rolf D. Beutler, Hochst/Hummetroth (DE); Karl-Heinz Schmidt, Gomaringen (DE); Bettina Funke, Frankfurt (DE); Kirstin Becker, Frankfurt (DE)

Correspondence Address: COLLARD & ROE, P.C., 1077 NORTHERN BOULEVARD, ROSLYN, NY, 11576, US

(21) Appl. No.: 11/659,393

(22) PCT Filed: Jul. 26, 2005
(86) PCT No.: PCT/EP2005/008087

§ 371 Date: Feb. 5, 2007

(30) Foreign Application Priority Data
Aug. 26, 2004 (DE) 10 2004041 270.7

(51) Int. Cl. A61K 9/28; A61K 31/198; A61K 33/30; A61K 33/10; A61K 33/34; A61K 33/06; A61K 31/51; A61K 33/00; A61K 36/55; A61K 36/00; A61K 31/519; A61K 31/4415; A61K 31/7052; A61K 31/355; A61K 31/122; A61K 31/455; A61K 31/59

(52) U.S. Cl. 424/474; 514/565; 424/643; 424/715; 424/634; 424/682; 514/276; 424/600; 424/94.1; 424/776; 424/768; 514/249; 514/277; 514/52; 514/458; 514/356; 514/562; 514/167

(57) ABSTRACT

This invention relates to compositions with a capillary-active system with application-relevant differentiability and synergistic potential for use in mammals, especially humans.

The invention accordingly relates to a composition that has a capillary-active system C optionally containing a capillary activator system CA consisting of drugs that increase blood flow, stabilize the vascular wall, and/or dilate the blood vessels; and/or a capillary-protective system CP comprising drugs for endothelial stabilization, lipoprotein protection, and for stabilization of leukocytes/platelets; and/or a capillary energy supply system CE comprising redox systems and cofactors in energy provision and energy carriers.

This system C is combined with a selective action system S.

The invention also relates to the use of such compositions, in particular systemically or enterally, especially for the selective control, for example, of structural changes, functional disturbances of the target organs in question (hair, skin, cerebrum, skeleton, muscles, gastrointestinal tract, eyes) as supplements (food supplements, supplementary balanced diet, dietary components), or pharmaceutical agents.

### OBJECT OF THE APPLICATION

This invention relates to compositions with a capillary-active system with application-relevant differentiability and synergistic potential for use in mammals, especially humans.

The invention accordingly relates to a composition that has a capillary-active system C optionally containing a capillary activator system CA consisting of drugs that increase blood flow, stabilize the vascular wall, and/or dilate the blood vessels; and/or a capillary-protective system CP comprising drugs for endothelial stabilization, lipoprotein protection, and for stabilization of leukocytes/platelets; and/or a capillary energy supply system CE comprising redox systems and cofactors in energy provision and energy carriers.

This system C is combined with a selective action system S.

The invention also relates to the use of such compositions—in particular systemically or enterally—especially for the selective control, for example, of structural changes, functional disturbances of the target organs in question (hair, skin, cerebrum, skeleton, muscles, gastrointestinal tract, eyes) as supplements (food supplements, supplementary balanced diet, dietetics), or pharmaceutical agents.

### PRIOR ART

Drugs are used for the care of individual organs. They target the specific activity of individual substances especially to the organ, with transport/residence time being decided by themselves.

In addition, there are supplements in particular like food supplements (micronutrient preparations). On the one hand, they are products with a number of widely different constituents with which the broadest possible needs of the body are to be met (for example, multivitamin preparations). It is unclear here whether such a broad activity spectrum actually exists, or whether intended effects are not mutually cancelled because of the multiplicity of different active groups.

For this reason, products are also used with each having only one active ingredient for a specific application (e.g. monovitamin preparations). Thus, for the particular different substances, a corresponding system also has to be made available so that the intended purpose can also be achieved. This may also depend on a definite dosage form. Thus, for example, DE 43 26 698 C2 describes the use of a Vitamin B6/B12/folic acid combination in particular as an injection for the prevention/treatment of arteriosclerosis. The same combination according to DE 43 26 675 C2 is used to prevent nerve degenerative disorders and senile dementia.

Drugs containing folic acid, 5-methyltetrahydrofolic acid, and/or 5-methyltetrahydrofolic acid polyglutamate are also described in DE 100 22 510 A1 as food supplements.

DE 102 06 159 A1 discloses drugs containing special combinations of folic acid, Vitamin B6, B12, and their use to lower homocysteine levels.

A Vitamin C preparation is disclosed in DE 100 35 088 C2 that is in a special form, namely as vesicles or beads with a diameter of 0.2 mm.

DE 198 58 372 A1 relates to a vitamin preparation comprising a carrier material consisting of a sugar in powder form, onto which a vitamin is applied, and a binder in crystalline agglomerated form. This form of binder is intended to lead to rapid disintegration upon contact with a small amount of fluid.

Pharmaceutical preparations for oral or peroral administration are disclosed by DE 100 16 313 A1; they are in the form of a gel with retarded drug release in the gastrointestinal tract.

EP B 0 531 155 describes the used of currant juice with no fruit pit oil or unsaturated fatty acids, to promote monoamine oxidase inhibition.

DE GM 201 16 346 relates to a micronutrient combination product with definite amounts of vitamins such as 350 to 700 mg of Vitamin C, etc., and carotinoids (2-10 mg), that are in separate dosage forms such as Vitamin C film-coated tablets+separate carotinoid capsules. This is intended to overcome deficiency symptoms, and also in particular no intensification of side effects is to be caused with concomitant administration of drugs.

A special combination preparation to promote hair and skin/nail growth is described in EP B 0 796 080; it also has Vitamin C in relatively large amounts (15.61%) and other vitamins/constituents in specific proportions.

EP B 1 001 685 concerns food compositions containing carbohydrates, fats, and protein, as well as in particular a given amount of methionine/cysteine.

GB A 2 292 522 describes a multivitamin preparation that has proteins and a mixture of vitamins as well as emulsifiers, and that is suitable for bringing about a normally functioning immune system.

A sugar-coated tablet with vitamins such as Vitamins A and C, calcium pantothenate, etc. and methionine among others, is described in the 2004 Red List® under Preparation No. 84166.

As is apparent from this, either only individual active ingredients are used, or a plurality of them are combined, with recourse then in particular to formulation measures with regard to the active ingredients themselves, or with regard to the accompanying materials combined with them, to regulate the stability, disintegration capability, or the rate of release. Such measures therefore also relate to the physical properties of the dosage form itself and are thus also greatly dependent on the active ingredients used. Therefore, new or modified dosage forms have to be developed again and again, depending on the active ingredient, and these are reserved either only for one ingredient and/or for a special group of ingredients, or a large number of different ingredients with no selective differentiability, i.e. without the existence of a general concept of use.

### PURPOSE OF THIS INVENTION

It is therefore the purpose of this invention to correct the deficiencies described above. A concept is to be developed by which a multidifferentiable drug transport occurs, in particular by a systemic or enteral path, with an economical, fast, and especially selective transfer of active ingredients to targets or target organs for which they are intended, using a single system—or module—without the necessity of developing a pharmaceutical formulation of their own in each case for the particular active ingredients.

### Accomplishment of the Task

This objective is reached pursuant to the invention by making available, in addition to a specific active system S, a multidifferentiable capillary-active system C comprising at least one system chosen from between a capillary activator system CA, a capillary-protective system CP, and/or a capillary energy supply system CE.

A generally usable module is thus obtained according to the invention that can be used in many ways depending on the desired target organ.

It is possible using such a system to achieve elevated efficiency in use, particular on the basis of faster and more economical transport by using the described capillary-active system C. This can also make it possible for the active drugs to act more economically on the target site (target organ), so that the applied dose can also be lowered in case of systemic or enteral administration.

All in all, efficacy is multifunctionally/selectively controlled as a function of the target (organ). It is thus possible to control or treat effectively and independently of each other the skin/appendages (hair, nails), or the keratinous system, or the immunologic and cerebral systems, as well as the skeletal system, muscles, gastrointestinal tract, or the eye. The invention thus makes available for the first time a system that is directed not at the special adaptation and processing of the specific active drugs, but instead, independently of them, allows improved supply and/or control of needy target organs, independently of each other, by activating the capillary system.

The composition pursuant to the invention is thus independent of the dosage form and is preferably designed for enteral, systemic, and particularly oral use.

### DESCRIPTION OF THE INVENTION

The invention in particular relates to a composition with application-relevant drug differentiability that is characterized by the fact that it has
I.) a capillary-active system C that comprises one or more systems selected from a
   a) capillary activator system CA comprising one or more drugs that are selected from one or more of the groups of
      CA 1) drugs that increase blood flow;
      CA 2) drugs that stabilize the vascular wall;
      CA 3) drugs that dilate blood vessels; and/or a
   b) capillary-protective system CP comprising one or more drugs selected from one or more of the groups of
      CP 1) drugs for endothelial stabilization;
      CP 2) drugs for lipoprotein protection;
      CP 3) drugs for leukocyte/platelet stabilization; and/or a
   c) capillary energy supply system CE comprising one or more drugs that are selected from one or more of the groups of
      CE 1) redox systems in energy provision;
      CE 2) cofactors in energy provision;
      CE 3) energy carriers; and
II.) a specific active system S selected from drugs of the group comprising drugs S1 that affect skin and appendages, cerebrally active drugs S2, immunologically active drugs S3, drugs S4 that affect bones, substances S5 that affect muscle, substances S6 that affect the gastrointestinal tract, drugs S7 that affect the eye, and contains
III.) 0.1 to 90% of additives, in particular galenicals/pharmaceutical additives.

Using such a composition, in particular for systemic or enteral use, it is possible in each case to produce application-relevant activity, especially in combination with given substances or groups of substances (drugs, nutrients, pharmaceutical ingredients) that is very much more selective/effective than the described multivitamin-nutrient preparations, and very much more effective than monofunctional products, since increased capillary activity and a heightened demand situation are developed by the capillary-active system C. Because of this the active substances can be transported specifically to a greater extent and improved activity can be realized. This was surprising in particular because individual components of the capillary-active system C were in fact known in themselves, but it could not have been expected that different target organs can be controlled selectively with the special combination with heightened demand.

The widest variety of dosage forms can be used. Among them in particular are oral drugs such as coated tablets, plain tablets, capsules, etc. The composition pursuant to the invention is thus suitable for systemic or enteral administration. Surprisingly, it is not necessary here to develop a special dosage form depending on the specific drug(s), since an improvement of the efficiency of different specific drugs of the system S is generally possible because of the active system C.

The specific drugs here are preferably selected from
S 1) substances that affect keratin, in particular skin and appendages;
S 2) cerebrally active substances;
S 3) immunologically active substances;
S 4) substances that affect bones;
S 5) substances that affect muscle;
S 6) gastrointestinally active substances;
S 7) drugs active on the eyes.

The compositions are therefore particularly suitable for controlling structural changes, disturbances of the target organs involved, and are suitable for systemic or enteral, and especially systemic oral administration.

The individual systems of the C group can be present in the composition pursuant to the invention in an amount of 0.001 to 80%, preferably 0.001 to 50%, and in particular 0.01 to 25%, each based on CA, CP, or CE, wherein the total of CA+CP+CE may be up to 98%, preferably up to 80%; 0 to 90%, preferably 0 to 65%, especially 0.01 to 45% additives, and 0.001 to 50%, especially 0.001 to 25% specific system S.

The quantity data refer to the total weight of the composition.

Thus, 2 systems are linked to one another in an especially effective manner with the composition pursuant to the invention, together with additives, namely the general capillary-active system C, with capillary activator CA, capillary protector CP, and/or capillary energy supply system CE, and the specific active system S, with the appropriate active substances for skin/hair/nails, bones, muscle, digestion, eyes, immune system, brain.

The collaboration of the two systems C+S is illustrated in the diagram below:

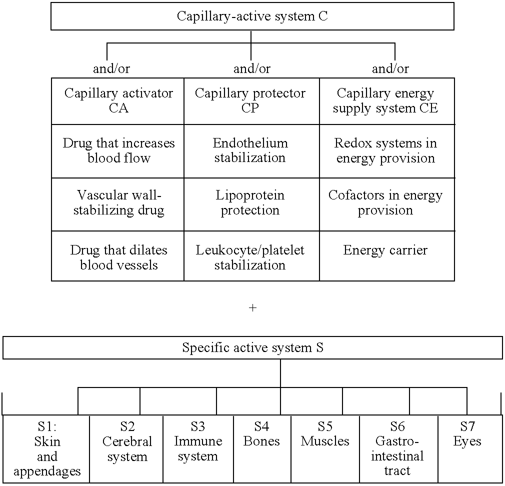

(+ Additives)

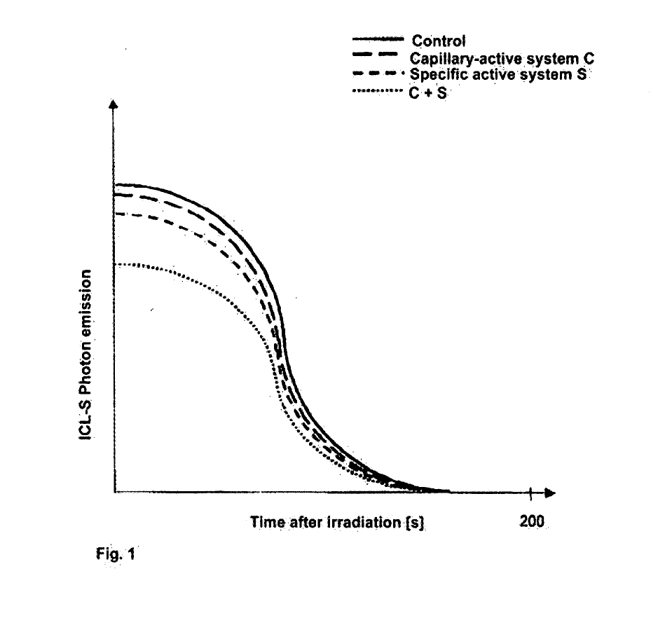

### DESCRIPTION OF FIG. 1

FIG. 1 shows the special efficacy of a composition pursuant to the invention versus comparison compositions, with reference to an ICL-S [Induced ChemiLuminescence of Human Skin] measurement.

### MORE DETAILED EXPLANATION AND PREFERRED EMBODIMENTS OF THE INVENTION

### I) Capillary-Active System C

The composition pursuant to the invention comprises a capillary-active system C that has one or more, or at least one, system selected from I a), I b), I c) as described

The composition may therefore have a capillary activator system CA or a capillary-protective system CP or a capillary energy supply system CE or combinations of 2 or 3 representatives of this group. In particular it contains a capillary activator system CA.

The capillary-active system C preferably comprises a system CA together with a system CP. In another preferred embodiment there is a combination of CA, CP, and CE, or of CA and CE.

It has been found that the capillary activator system CA, because of its blood flow-promoting properties, causes activation of this system. The CA system is thus agonistically effective and can even initiate superadditive effects, up to synergistic effects as the case may be, with combinations of drugs from the group CA 1)+CA 2); CA 1)+CA 3); CA 2)+CA 3), or CA 1)+2)+3).

The CP system in particular leads to endothelial stabilization, lipoprotein protection, and leukocyte/platelet stabilization, whereby harmful effects, for example by oxidants, are additionally suppressed and regeneration of the endothelium is thus made possible. The CP system can thus be called antagonistic overall.

The CE system, finally, can lead to additional reinforcement by agonistic activities.

A superadditive to synergistic effect, as the case may be, can also be generated in particular by a combination of the agonistic system CA (CA1/2/3) and the antagonistic system CP, or with the antagonistic system CP and the system CE, or by combination of CA/CP/CE with a desired active system S. This can be demonstrated by suitable methods [ICL-S measurement, imaging procedures (capillary angiography, thermography), oximetry], or histamine response. The special efficacy of a composition pursuant to the invention over comparison compositions is shown below by an example of use, and in FIG. 1 by means of ICL-S measurement.

The drugs to be used for the individual CA, CP, CE systems with the described properties are familiar to one skilled in the art.

The following substances, also including preferred substances especially, can be mentioned below by way of example:

Capillary Activator System CA:

Grape leaf extract, rutin, aescin, horse chestnut extract, Butcher's broom extract, iron carbonate/fumarate/diphosphate/lactate/saccharate/gluconate/sulfate/citrate [e.g. in particular in an amount corresponding to 6 mg of iron (alone or in combination)], ginkgo extract, especially NO releasers such as arginine and its salts like L-arginine hydrochloride. The latter or its precursors can be present in particular in an amount corresponding to 250 mg of L-arginine.

### Capillary-Protective System CP:

Tocopherol/acetate (Vitamin E), anthocyans, α-liponic acid, folic acid (B₉), pyridoxine HCl (B₆), cyanocobalamine (B₁₂), borage oil, linseed oil, grape seed oil, black currant seed oil, evening primrose oil, salmon oil, black caraway seed oil (precursors of Q-3 and Q-6 fatty acids).

### Capillary Energy Supply System CE:

Nicotinic acid (niacin) and its suitable derivatives such as especially nicotinamide (B₃), magnesium oxide/citrate/carbonate/chloride/gluconate/phosphate/lactate/sulfate, riboflavin (B₂), L-carnitine, Q₁₀, caffeine, glucose.

Especially preferred embodiments are characterized by the fact that they have a capillary activator system CA and a capillary-protective system CP or an energy supply system CE; or also those that have a capillary-protective system CP and a capillary energy supply system CE besides the capillary activator system CA.

Very highly preferred drugs for the CA system are arginine and its salts (for example particularly as described), ginkgo extract or combinations thereof; those for the CP system are folic acid, cyanocobalamine, pyridoxine HCl (B₆), tocopherol acetate or combinations thereof; and those for the CE system are nicotinic acid (niacin), and its suitable derivatives like nicotinamide in particular, and magnesium oxide; coenzyme Q10, riboflavin or combinations thereof.

The amounts of each of the systems CA, CP, CE are preferably 0.001 to 80%, based on the total weight of the composition. Especially preferred amounts are from 0.01 to 50%, particularly 0.01 to 35%.

All quantity and percentage data refer to the weight of the composition if not otherwise indicated.

Very highly preferred are also compositions of the type described wherein the capillary activator system CA has one or more drugs selected from ginkgo extract, Butcher's broom extract, L-arginine hydrochloride (for example in amounts as described); the capillary-protective system CP has one or more drugs selected from folic acid, pyridoxine hydrochloride, cyanocobalamine, tocopheryl acetate, oils containing omega-3 and omega-6 fatty acids, and the capillary energy supply system CE has one or more drugs selected from nicotinic acid and its derivatives, especially nicotinamide, magnesium oxide, caffeine, glucose, and Q10.

Especially preferred are compositions wherein the capillary-active system C has ginkgo extract, folic acid, and magnesium oxide.

Some examples of drugs for the active systems CA, CP, and CE are indicated below in the specific amounts for each, for example such as:

CA: Ginkgo extract, for example corresponding to 5 g of drug
Grape leaf extract, for example corresponding to 2 g of drug
Rutin: 1000 mg
Aescin: 100 mg
Horse chestnut extract: corresponding to 100 mg of aescin
Butcher's broom extract: corresponding to 7-11 mg of ruscogenine L-Arginine (for example xHCl) corresponding to 250 mg of L-arginine
Iron (e.g. xcarbonate) corresponding to 6 mg of iron
CP: Tocopheryl acetate: corresponding to 42 mg of Vitamin E
Oil containing omega-3 fatty acids: corresponding to at least 90 mg of omega-3 fatty acids;
Oil containing omega-6 fatty acids: corresponding to at least 360 mg of omega-6 fatty acids;
Folic acid: 0.2 to 5 mg, preferably 0.2 to 0.8 mg, especially 0.4 mg;
Vitamin B6: 2.0 to 25 mg; preferably 2.0 to 10 mg, especially up to 6 mg, and with very great preference 2 mg
Vitamin B12: 0.003 to 0.6 mg, particularly up to 0.1 mg, especially 0.006 mg;

Preferred Quantity Ranges here are:
Folic acid 0.4-1.2 mg; Folic acid:Vitamin B₆=1:1-12
Vitamin B₆ 1.2-4.5 mg: Vitamin B₁₂=1:0.0025-0.0225
Vitamin B₁₂ 0.003-0.009 mg; Vitamin B₁₂:Vitamin B₆=1:40-500
For mixtures of these substances, the ratio can be
Folic acid:Vitamin B₆=1:5
Folic acid:Vitamin B₁₂=1:0.015
Vitamin B₁₂:Vitamin B₆=1:333.
CE: Nicotinamide: 30 mg
Magnesium oxide/carbonate: corresponding to 150 mg of magnesium
Riboflavin: 4 mg
L-Carnithine (500 mg), caffeine, glucose (500 mg)

Very highly preferred from the CA group is L-arginine hydrochloride, for example as especially described.

From the CP group, the combination of folic acid, Vitamin B6, and Vitamin B12 in the ratio by weight of 1:5.0:0.15 is especially preferred.

From the CE group, nicotinic acid/its derivatives, especially nicotinamide and magnesium salts or combinations thereof are chosen in particular.

In a very highly and especially suitable embodiment, a CA system is combined with a CP system with folic acid, Vitamin B6, or Vitamin 12.

Another suitable embodiment provides for the CA, CP combination described above together with nicotinic acid, its derivatives, especially nicotinamide, magnesium salt, or combinations thereof.

The individual substances from the named groups are known in themselves or can be prepared by methods familiar to one skilled in the art.

### II) Specific Active System S

As explained, the composition pursuant to the invention, besides the capillary-active system C, also has a specific active system S selected from drugs of the group comprising drugs S1 that affect skin and appendages (hair, nails), cerebrally active drugs S2, immunologically active drugs S3, drugs S4 affecting bones, drugs S5 active on muscles, drugs S6 active on the gastrointestinal tract, or drugs S7 active on the eyes.

Especially preferred for this are compositions that have a capillary activator system CA and a capillary-protective system CP, and a specific active system S or a specific active system S2 or S3; or those that also have a capillary energy transfer system CE in addition thereto.

The active ingredients suitable for each of the aforementioned individual groups and the amounts suitable for their use are known to those active in the field of producing such compositions for systemic or enteral products, particularly food products, and can accordingly be singled out and incorporated in the composition pursuant to the invention depending on the action target.

The following substances can be mentioned by way of example as preferred drugs for the active system S:

Skin and Appendages System S1:

Retinol, retinol acetate/palmitate (Vitamin A), thiamine nitrate (B₁), biotin, Ca pantothenate (B₅) (B vitamins), diatomaceous earth, silicic acid, zinc oxide/sulfate/gluconate, vegetable extracts such as algae, papaya, rough horsetail, camomile, aloe vera, amino acids such as cysteine, methionine, gamma-linolenic acid (GLA), linoleic acid; Vitamin C (ascorbic acid, calcium ascorbate/sodium ascorbate/potassium ascorbate).

### Cerebral System S2:

Vegetable extracts such as ginseng, brazil nut, pennywort, cohosh, amino acids such as glutamic acid, tyrosine, tryptophan, uridine; docosahexaenoic acid (DHA), phosphatidylserine, phosphatidylinositol, choline, lecithin.

Immunological System S3:

Vitamins such as carotinoids like lycopene, sodium selenate, vegetable extracts such as green tea, echinacea, schisandra, astragalus, reishi, shiitake, maitake, red clover, soy, cat's claw, grapeseed, broccoli, tomato, onion, cauliflower, citrus bioflavonoids, buckwheat, grapefruit, amino acids such as threonine, histidine, glutathione, superoxide dismutase.

### Skeleton System S4:

Substances from the Vitamin D group such as cholecalciferol, ergocalciferol, Vitamin K1 (phylloquinone), calcium salts such as calcium carbonate/citrate/chloride/gluconate/lactate/oxide, dicalcium phosphate/acid phosphate, fluorides such as potassium fluoride, sodium fluoride, trace elements such as copper carbonate/citrate/lysine complex/sulfate/gluconate, manganese carbonate, chloride, citrate, gluconate, sulfate, vegetable extracts such as soy isoflavones, alfalfa, barley, lambsquarters, amino acids such as lysine.

### Muscle System S5:

Inositol (B₈), amino acids such as leucine, isoleucine, valine.

### Gastrointestinal System S6:

α-Linolenic acid,
potassium salts such as potassium carbonate/bicarbonate/citrate/chloride/gluconate/lactate/phosphate, chromium salts such as chromium chloride/picolinate/sulfate.

### Eye System S7:

Carotinoids such as beta-/alpha-carotene, beta-cryptoxanthine, lutein, zeaxanthin, vegetable extracts such as red wine bioflavonoids, elder bioflavonoids, quercetin; amino acids such as taurine.

It is also particularly beneficial for the composition pursuant to the invention to have from 1 to 5, preferably from 1 to 3 compounds of the CA system; 1 to 5, preferably 1 to 3 compounds of the CP system; 1 to 5, preferably 1 to 3 compounds of the CE system; and/or 1 to 5, preferably 1 to 3 compounds of the S system.

Especially preferred embodiments of such systems pursuant to the invention are compositions in which one or more substances selected from cysteine, methionine, biotin, zinc oxide, thiamine nitrate (B1), calcium pantothenate (B5), and diatomaceous earth is/are included as drugs of the S1 system; ginseng, Brazil nuts, cohosh, amino acids as discussed or mixtures thereof are included as drugs of the S2 system; vegetable extracts such as green tea, echinacea, schisandra, astragalus, reishi, shiitake, maitake, red clover, soy, cat's claw, grapeseed, broccoli, tomato, onion, cauliflower, citrus bioflavonoids, buckwheat, and grapefruit is/are included as drugs of the S3 system; or one or more carotinoids such as beta-/alpha-carotene, beta-cryptoxanthine, lutein, zeaxanthin, vegetable extracts such as red wine bioflavonoids, elder bioflavonoids, quercetin; amino acids such as taurine is/are included as drugs of the eye system S7, and also compositions wherein the activator system CA is L-arginine hydrochloride, the protector system CP includes drugs from the CP group: folic acid; pyridoxine HCl (B6); cyanocobalamine (B12); tocopherol (E); grapeseed oil, and/or currant seed oil, and the drug(s) for the CE group are selected from Q10, nicotinamide (niacin, B3), or mixtures thereof.

Also preferred are compositions with a CA+CP system or CA+CE+CP+S5 or S6.

Especially preferred are compositions with drugs of the S1 or S2 or S3 group that have 0.1 to 10% additives.

The individual active ingredients of the S group, their preparation and their suitable quantities, are known in themselves.

Pharmacologically active ingredients are also possible that can be assigned to the individual groups S1 to S7. Examples of these (other known substances are also possible) that may be mentioned are:

Hair: finasteride, minoxidil
Skin: isotretinoin, prednisolone
Cerebrum: amantadin, dopamine
Skeleton: calcitonin, risedronic acid
Muscles: dantrolen sodium, tetrazepam
Eyes: acetazolamide, pilocarpine
Gastrointestinal tract: famotidine, metoclopramide

The drugs named are mentioned only by way of example and do not exclude others. One skilled in the art may be able to incorporate the desired substances.

### III Additives

The composition pursuant to the invention may have additives in an amount of 0.1 to 90 wt. %, or preferably 1 to 90 wt. %, especially 0.5 to 80 wt. %, primarily 2 to 80 wt. %, or 1 to 50 wt. %, particularly 2 to 50 wt. %, based on the total weight of the composition, which result from the manufacture of the mentioned dosage forms. Among them in particular, especially for enteral, especially oral administration, are: excipients in liquid or solid form as well as pharmaceutically acceptable additives or added substances that are known for the production of supplements such as dietary supplements, supplementary balanced diet, or dietetics.

The excipient for solid oral forms of administration (tablets, capsules, coated tablets) ordinarily consists of conventional, pharmaceutically acceptable tableting auxiliaries such as disintegrants, fillers, flow aids, or other dissolution aids such as citric acid, bicarbonate (for effervescent tablets, for example), liquids such as water, (fruit) juices, carbohydrate-protein mixtures (for edible bars), or for example of soft gelatin capsules in which the composition is incorporated. Specially designed dosage forms are not necessary here; the methods and products known from the prior art are used in each case.

### Manufacture

The composition pursuant to the invention for the described administration, especially enteral administration, is manufactured by known methods, by producing the system C containing CA, CP, CE, or combinations thereof by mixing the individual desired substances, and then mixing this with the system S, selected from S1, S2, S3, S4, S5, S6, or S7 in a suitable manner, optionally using additives suitable in each case for the desired oral dosage form such as excipients, fillers, tableting aids, as mentioned, and homogenizing. Methods for producing such foods or pharmaceuticals are described in textbooks, for example, and are generally known.

Depending on the active ingredient of the S group, pharmaceutical preparations or supplements can be obtained as described in this way.

The composition for that matter can also be part of a drug in the form of a preparation such as edible bars, etc. For this purpose, food components can be chosen from amino acids, carbohydrates, or fats that are suitable for human or animal consumption, for example carbohydrate-protein mixtures. Examples of known individual components are fruit juice, nectar, or fruit jam, such as apple juice, orange juice, or apple sauce. Other known individual components are grain products such as wheat or rye flour, oatmeal, corn syrup, lactoprotein, whey, lecithin, lactose. Depending on the choice of such representatives of the mentioned individual components, edible bars, liquids, drinks, for example power drinks (preferably nonviscous/aqueous-syrupy), effervescent tablets, or other balanced diet supplements of this type can be produced.

To this end, the composition prepared as described is mixed with one or more food components by known methods, and the desired form is obtained by conventional production processes.

### Administration

The compositions pursuant to the invention are intended particularly for systemic or enteral administration. Oral dosage forms of compositions from the active drug system C+system S are especially suitable.

Particularly suitable dosage forms are gelatin capsules, for example soft gelatin capsules, and tablets.

Because of the particular combination of the composition pursuant to the invention, products are obtained that are stable overall, whose mode of action can be produced independently of the dosage form.

In particular, this composition can be used in case of structural changes and functional disturbances of the organs in question, especially of the hair, nails, skin, eyes, muscles, gastrointestinal tract, cerebral capabilities, skeleton, immunological system, as a supplement (non-therapeutic) such as a food supplement, dietary component, balanced diet supplement, or as a pharmaceutical agent or for the preparation of a pharmaceutical agent.

This composition can be used as mentioned for the systemic, especially oral, treatment or prevention of structural changes and functional disturbances of the target organs in question such as the skin, nails, hair, cerebrum, skeleton, muscles, eyes, and of the immunological system or gastrointestinal tract of mammals, particularly humans.

To produce capsules or tablets, auxiliaries customary for the purpose, such as lactose, sucrose, sorbitol, talc, stearic acid, magnesium stearate, gum arabic, potato starch, gelatins, PVP, glycerin, hydroxypropylmethylcellulose, maltodextrin, preservatives, and customary materials for coatings such as PEG 6000, cornstarch, sugars, talc, and dyes can be used in known ways. When oral administration is used, the described composition can be mixed, for example, with lactose, sucrose, powdered starch, cellulose esters, or alkanoic acids, cellulose alkyl ethers, talc, stearic acid, magnesium stearate, magnesium oxide, sodium and calcium salts of phosphoric and sulfuric acids, gelatins, acacia gum, sodium alginate, glycols, polyvinylpyrrolidone, and/or polyvinyl alcohol, and can then be tableted or encapsulated.

The additives and tableting methods are widely known, and are described, for example, in the current European Pharmacopoeia. The structural changes and functional disturbances may be related in particular to debilities, for example after illnesses or special stress, or to aging or deficiencies.

The quantity of drug administered and the dosage schedule for prevention or treatment of a condition as described above with the composition pursuant to the invention for a medical indication depend on multiple factors, including age, weight, sex, and condition of the patient, severity of the problem, the route of administration, and the particular compound used, and may therefore vary extremely widely. The necessary or desired dose can be spread over one or more times daily, especially 1 to 3 times.

When administered as a supplement, the usual customary amounts are suggested, which can be spread over one or more times, especially 1 to 3 times or 1 to 2 times daily. These can be in solid form, for example as tablets, capsules, coated tablets, or edible bars, or they can be a corresponding amount dissolved in juices.

### EXAMPLES

The invention will be described in further detail with reference to the following Examples 1 to 4. The efficacy of the composition pursuant to the invention is shown in Example 5 below.

### Example 1

### Coated Tablet with Active System S1 (Skin)

A coated tablet pursuant to the invention was obtained by mixing the following substances and compressing them under the usual conditions:

### I. Capillary-Active System C:

1) Drug from the CA group: L-Arginine hydrochloride:
2) Drugs from the CP group: Folic acid, pyridoxine HCl (B6); cyanocobalamine (B12); tocopherol (E); grapeseed oil;
3) Drug from the group CE: Q10:

### II: Specific Active System 3

S1: Thiamine nitrate (B1), calcium pantothenate (B5), diatomaceous earth,

### III: Additives: Soy Lecithin, Soybean Oil, Gelatin, Glycerin

### Example 2

### Tablet with Active System S1 (Hair)

A tablet was obtained by mixing the individual constituents and additives by known methods as follows:

I. Capillary-active system C

1) CA: L-Arginine hydrochloride

2) CP: Red currant seed oil

3) CE: Nicotinamide (niacin, B3)

II: Specific active system S

S1: Cysteine, methionine, biotin, zinc oxide

III: Additives: Lactose, magnesium stearate, Aerosil

### Example 3

### Hard Gelatin Capsule with Active System S3 (Skeleton)

A hard gelatin capsule was obtained by known methods with the following substances:

I. Capillary-active system C
CA: L-Arginine hydrochloride
   II. Specific active system S
S3: Cholecalciferol (D3, phylloquinone (K1), calcium carbonate, copper carbonate
   III. Additives: Gelatin, magnesium stearate, talc, Aerosil

### Example 4

### Soft Gelatin Capsule, Active System S1

A soft gelatin capsule was obtained by known methods by mixing the individual constituents, as follows:

I. Capillary-active system C
CA: L-Arginine hydrochloride;
CP: Vitamin E, grapeseed oil, linseed oil;
CE: Coenzyme Q10; magnesium salt, nicotinamide;
   II. Vitamin A palmitate; biotin; zinc salt; Vitamin C ascorbate,
   III. Additives: Glycerin, gelatin, sorbitol.

### Example 5

### Applied Example

60 chosen test volunteers with healthy skin, 15 in each group, were given a combination of vital substances as described in the following table, or only the combination C or S according to the table, or placebo (Group C+S, C, S, Control) for a period of 6 months, a soft gelatin capsule in the morning and in the evening, with the substances indicated below.

The superiority of the combination of a specific active system “S” with a capillary-active system “C” is shown impressively by this applied example. In this procedure, a skin area of volunteer subjects is irradiated with ultraviolet light and the photon emission from the irradiated skin is then measured by “Single Photon Counting.” The lower the photon emission, the better is the protection of the skin against oxidative damage. After taking the particular composition C, or S, or C+S being examined, an effect is achieved that can be demonstrated measurably by UV light protection. The protective properties are surprisingly more pronounced with the C+S combination than with the individual components C, S, and the control group. This fact serves as proof of efficacy for the superior formulation according to the composition pursuant to the invention, which can also be called the Micro Nutri-Targeting system.

### Description of Methods:

The so-called ICL-S (induced chemiluminescence of human skin) was measured on the backs of the test subjects with healthy skin selected before beginning the study when they started to take the particular composition, and when they stopped taking it.

For the irradiation, a dome of liquid optical waveguides with a centrally located waveguide is positioned at an optimal distance from the skin without touching it, and at an optimal angle. The optical waveguides are in a measuring head that is pressed firmly against the skin. This arrangement permits a defined photon transfer from the skin to the detector and compensates for influences such as sweating or motions of the test subject.

Each person was irradiated at four study areas for two minutes with UVA (320≦λ≦400 nm) (energy of irradiation 10 mW/cm², λmax=350 nm). Immediately after this UV stress, the photon emission of the skin was measured by single-photon counting over a period of 200 s. The amount of photons released then correlates with the “oxidative stress” initiated by the UVA radiation [Sauermann G, Mei W P, Hoppe U, Stab F; Ultraweak photon emission in vivo: Influence of topically applied antioxidants on human skin, Methods Enzymol 199; 300:419-428].

The average of the integrated photon emission and the average kinetics of decay of the photons emitted from the skin were calculated before and after taking the combination, and were tested for significant differences.

Differences in skin coloring were taken into consideration computationally.

It was proven during this study that intervention with the combinations C+S pursuant to the invention leads to strengthening of the antioxidative protective function of the skin. Oxidized metabolites were not detected indirectly here, as is otherwise customary; instead, there was a direct measurement of the UV-induced chemiluminescence of the skin of the individual subjects. In this way, functionally relevant results were obtained by a physical method of measurement that permitted an immediate conclusion about the protection of the skin against damage from exogenous noxae. This improvement of the protective function of the skin is important not only in the sense of a dermal anti-aging effect, but also in the sense of preventing many age-dependent diseases of the skin that are associated with oxidative stresses.

Since damage occurs to mitochondrial DNA as a result of UVA stress from sunlight, that can not only persist for years, but whose content in the skin may rise further even without further irradiation from a mechanism once induced, antioxidants are recommended to avoid photo-aging in the sense of permanent treatment and not only for the time of exposure to the sun [GD

Symposium: Effects of dermacosmetics, Dermotopics 1, 2002; 93:9-12].

Of course the use of antioxidants in no case should lead to misguided overexposure to the sun.

The particular combination C, or S, or C+S used contained the following substances (micronutrients), listed by type and quantity:

| | Substance | | |
|---|---|---|---|
| Module | (micronutrient) | Daily dose | |
| C | Capillary activator system CA | | |
| Capillary-active | Blood vessel dilation, blood flow | | |
| system | increase | | |
| | L-Arginine | 250 | mg |
| | Capillary protector system CP | | |
| | Endothelial stabilization, blood vessel | | |
| | protection, lipoprotein protection | | |
| | Vitamin E | 42 | mg |
| | Grapeseed oil contains | 514 | mg |
| | at least Ω-6 fatty acids | 360 | mg |
| | Linseed oil contains | 178.8 | mg |
| | at least Ω-3 fatty acids | 90.0 | mg |
| | Capillary energy supply system CE | | |
| | Redox systems and cofactors in energy | | |
| | provision, energy carriers | | |
| | Coenzyme Q₁₀ | 5 | mg |
| | Magnesium | 300 | mg |
| | Niacin | 39 | mg |
| S | Specific active system S | | |
| Specific active | Specific active system, skin S | | |
| system | Vitamin A | 0.8 | mg |
| | Biotin | 0.18 | mg |
| | Zinc | 9.0 | mg |
| | Vitamin C | 100 | mg |

The ICL values obtained are shown in FIG. 1 as a function of time.

As is apparent from this, the combination of vital substances from C+S pursuant to the invention leads to a surprising strengthening of the antioxidative protective function of the skin. An unexpected superadditive effect is found here. This is all the more beneficial since the damage to mitochondrial DNA as a result of UVA stress from sunlight can persist for years, and furthermore its content in the skin may rise further even without further irradiation from a once-induced mechanism. Antioxidants are therefore especially desirable to avoid photo-aging, particularly also in the sense of permanent treatment and not only for the time of exposure to the sun.

1. Composition with application-relevant drug differentiability, wherein it has
I.) a capillary-active system C that comprises one or more systems selected from a
a) capillary activator system CA comprising one or more drugs that are selected from one or more of the groups of
CA 1) drugs that increase blood flow;
CA 2) drugs that stabilize the vascular wall;
CA 3) drugs that dilate blood vessels; and/or a
b) capillary-protective system CP comprising one or more drugs selected from one or more of the groups of
CP 1) drugs for endothelial stabilization;
CP 2) drugs for lipoprotein protection;
CP 3) drugs for leukocyte/platelet stabilization; and/or a
c) capillary energy supply system CE comprising one or more drugs that are selected from one or more of the groups of
CE 1) redox systems in energy provision;
CE 2) cofactors in energy provision;
CE 3) energy carriers; and
II.) a specific active system S selected from drugs of the group comprising drugs S1 that affect skin and appendages, cerebrally active drugs S2, immunologically active drugs S3, drugs S4 that affect bones, substances S5 that affect muscle, substances S6 that affect the gastrointestinal tract, drugs S7 that affect the eye, and contains
III.) 0.1 to 90% of additives.

2. Composition according to claim 1, wherein the capillary-active system C has a capillary activator system CA.

3. Composition according to claim 1, wherein the capillary-active system C has a capillary protector system CP.

4. Composition according to claim 1, wherein it has a capillary energy supply system CE.

5. Composition according to claim 1, wherein it has a capillary activator system CA and a capillary protector system CP.

6. Composition according to claim 5, wherein that it also has a capillary energy supply system CE.

7. Composition according to claim 1, wherein a capillary activator system CA and a capillary protector system CP and a specific active system S1 or specific active system S2 or S3 are included; or in addition thereto, a capillary energy carrier system CE is also included.

8. Composition according to claim 1, wherein the capillary activator system CA has one or more drugs selected from ginkgo extract, Butcher's broom extract, L-arginine hydrochloride; the capillary-protective system CP has one or more drugs selected from folic acid, pyridoxine hydrochloride, cyanocobalamine, tocopheryl acetate, oils containing omega-3 and omega-6 fatty acids, and the capillary energy supply system CE has one or more drugs selected from nicotinic acid and its derivatives, especially nicotinamide, magnesium oxide, Coenzyme Q10, caffeine, and glucose.

9. Composition according to claim 8, wherein the capillary-active system C has ginkgo extract, folic acid, and magnesium oxide.

10. Composition according to claim 1, wherein the system S1 contains as drugs one or more substances selected from cysteine, methionine, biotin, zinc oxide, thiamine nitrate (B1), calcium pantothenate (B5), diatomaceous earth; the system S2 contains as drugs ginseng, brazil nut, pennywort, amino acids such as glutamic acid, tyrosine, tryptophan, uridine, or mixtures thereof; and the system S3 contains as drugs vegetable extracts such as green tea, echinacea, schisandra, astragalus, reishi, shiitake, maitake, red clover, soy, cat's claw, grapeseed, broccoli, tomato, onion, cauliflower, citrus bioflavonoids, buckwheat, grapefruit, or it contains as drugs of the eye system S7 one or more carotinoids such as beta-/alpha-carotene, beta-cryptoxanthine, lutein, zeaxanthin, vegetable extracts such as red wine bioflavonoids, elder bioflavonoids, quercetin; amino acids such as taurine.

11. Composition according to claim 1, wherein it has additives customary for drug or pharmaceutical formulations in an amount of 0.1 to 90 wt. %, especially 2-80 wt. %.

12. Composition according to claim 1, wherein it can be administered systemically, especially enterally.

13. Composition according to claim 1, wherein it is in a dosage form suitable for systemic or oral administration.

14. Composition according to claim 13, wherein it is in the form of a tablet, capsule, coated tablet, or a liquid, or of an edible bar, effervescent tablet, or gelatin capsule, in a suitable excipient base selected from tableting aids, water, fruit juices, or carbohydrate-protein mixtures.

15. Use of a composition according to claim 1 as a supplement for structural changes and functional disturbances of the target organs in question, especially hair, nails, skin, cerebrum, skeleton, muscles, eyes, immunological system, and gastrointestinal tract.

16. Use according to claim 15, wherein the supplement is a food supplement, a dietary component, or supplementary balanced diet.

17. Use according to claim 15, wherein the structural changes and functional disturbances are related to debilities, aging symptoms, or deficiency symptoms.

18. Use according to claim 15, wherein the supplement is in the form of a coated tablet, a capsule, a plain tablet, a beverage, or an edible bar.

19. Use of a composition according to claim 1 to prepare a drug in the form of a coated tablet, capsule, plain tablet, a beverage, or an edible bar to control structural changes and functional disturbances, especially debilities and aging symptoms of the hair, nails, skin, cerebral capabilities, skeleton, immunological system, muscles, gastrointestinal tract, or eyes.

20. Use according to claim 19 as a pharmaceutical agent.
